(19) United States
(12) Patent Application Publication
(10) Pub. No.: US 2006/0069382 A1
(43) Pub. Date: Mar. 30, 2006

(54) Delivery device

(75) Inventor: Per Elgaard Pedersen, Haslev (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/243,773

(22) Filed: Oct. 5, 2005

Related U.S. Application Data
(63) Continuation of application No. PCT/DK04/00218, filed on Mar. 29, 2004.

(30) Foreign Application Priority Data
Apr. 11, 2003 (DK) PA 2003 00562
Sep. 3, 2003 (DK) PA 2003 01259

(51) Int. Cl. A61K 9/22

(52) U.S. Cl. 604/890.1

(57) ABSTRACT

This invention relates to a delivery device of the bleeding hole type, where a primary drive fluid, e.g. silicon oil, is used to expel a secondary fluid, e.g. a drug, contained in a reservoir. To provide a desired drug flow rate, the primary fluid is forced from a first reservoir through a flow restrictor into a second reservoir displacing a portion of the drug reservoir, thereby expelling the drug from its reservoir. The idea is to provide a drive fluid outlet, i.e. a flow restrictor inlet, which protrudes into the first reservoir. By this arrangement the amount of particles and air-bubbles entering the narrow flow restrictor will be reduced. The reduction is achieved because particles and air-bubbles will normally concentrate in the top or bottom of the reservoir, whereas the protrusion will primarily connect to the centre of the first reservoir.

### CROSS-REFERENCE TO RELATED PATENT APPLICATIONS

This patent application is a continuation of PCT Patent Application No. DK2004/000218, filed Mar. 29, 2004 and claims the benefit of U.S. Provisional Patent Application Nos. 60/470,145 filed May 13, 2003, and 60/503,699 filed Sep. 17, 2003 and Danish Patent Application Nos. PA 2003 00562 filed Apr. 11, 2003 and PA 2003 01259 filed Sep. 3, 2003, the contents of which are fully incorporated herewith by reference.

### FIELD OF THE INVENTION

The present invention relates to means providing improved functionality and reliability for a fluid delivery device. Such delivery devices are suitable in particular for in situ administration of a therapeutic drug preparation over a prolonged period of time, however, the delivery devices may also be used in areas such as biochemistry, microbiology and chemical analysis.

### BACKGROUND OF THE INVENTION

In the disclosure of the present invention reference is mostly made to the treatment of diabetes by injection or infusion of insulin, however, this is only a preferred use of the present invention.

Diabetes mellitus is the common name for at least 2 different diseases, one characterised by immune system mediated specific pancreatic beta cell destruction (insulin dependent diabetes mellitus (IDDM) or type 1 diabetes), and another characterised by decreased insulin sensitivity (insulin resistance) and/or a functional defect in beta cell function (non-insulin dependent diabetes mellitus (NIDDM) or type 2 diabetes).

The principal treatment of type 1 diabetes is straight forward substitution of the missing insulin secretion, whereas treatment of type 2 is more complicated. More specifically, in early stages of type 2 diabetes treatment a number of different types of drugs can be used, e.g. drugs which increase insulin sensitivity (ciglitazones), decrease hepatic glucose output (e.g. metformin), or reduce glucose uptake from the gut (alfa glucosidase inhibitors), as well as drugs which stimulate beta cell activity (e.g. sulfonylurea/meglitinides). However, the above-described deterioration is reflected in the fact that beta cell stimulators will eventually fail to stimulate the cell, and the patient has to be treated with insulin, either as mono therapy, or in combination with oral medication in order to improve glucose control.

Currently, there are two principal modes of daily insulin therapy, the first mode including syringes and insulin injection pens. These devices are simple to use and are relatively low in cost, but they require a needle stick at each injection, typically 3-4 times or more per day. The second mode is infusion pump therapy, which entails the purchase of a relatively expensive pump, for which reason the initial cost of the pump is a barrier to this type of therapy. Although more complex than syringes and pens, the pump offer the advantages of continuous infusion of insulin, precision in dosing and optionally programmable delivery profiles and user actuated bolus infusions in connections with meals.

Basically the infusion pump comprises means for allowing the contained insulin to be transferred to the body of the patient. These means may take any desirable form providing the desired function, but presently pump arrangements comprising a conveying arrangement connected to the reservoir (i.e. an outlet to be associated with needle infusion means) and including a pressure or suction generating device for feeding the liquid contained in the reservoir by pressure or suction application from the reservoir to the body are preferred for transferring the insulin contained in the reservoir to the patient. In this respect a number of different principles may be utilized, e.g. osmotic pumps as known from for example U.S. Pat. Nos. 4,340,048 and 4,552,561, piston pumps as known from for example U.S. Pat. No. 5,858,001, membrane pumps as known from for example U.S. Pat. No. 6,280,148, flow restrictor pumps (also known as bleeding hole pumps) as known from for example U.S. Pat. Nos. 2,605,765 and 5,957,895, and gas generating pumps as known from for example U.S. Pat. No. 5,527,288, which all in the last decades have been proposed for use in durable (refillable) and/or disposable (prefilled) drug infusion systems. Two of the above principles may be combined in a single pump, e.g. in U.S. Pat. No. 2,605,765 a bleeding hole pump is used to drive a piston pump which may thus be characterized as a “secondary” pump. As some of these principles may not be considered to be pumps in the traditional sense, it may be more appropriate to generally describe these devices as delivery means for fluid, however, in the following description the traditional term pump will be used.

Of the above pump principles, the present invention is especially useful for the bleeding hole type. Basically, this principle provides a means for establishing a flow of a fluid at a desired rate by applying a force to a liquid to thereby force the liquid through a flow restrictor, the flow rate being determined by the pressure generated on the fluid by the applied force, the flow resistance in the flow restrictor per se and the viscosity of the fluid. As the flow resistance is determined in combination by the structure of the flow restrictor (i.e. its configuration and dimensions) and the viscosity of the fluid, the term “flow resistance in the flow restrictor per se” refers to the former component. When in the following reference is made to the “flow resistance in the flow restrictor” this is to be understood as the “flow resistance in the flow restrictor per se”. For the purpose of expelling a drug from a reservoir, two variants of this principle have been described.

In a first variant the drug to be infused is contained in a reservoir in fluid communication with an outlet through a flow restrictor. When the drug is pressurized by an actuating (driving) force it is forced through the flow restrictor at a rate determined by the applied force, the flow resistance in the flow restrictor and the viscosity of the drug, see for example U.S. Pat. No. 5,957,895 which discloses an infusion device in which the driving force is provided by a drug reservoir formed between two Belleville springs, the flow restrictor being provided by the through-going channel of a capillary tube, or WO 02/15965 disclosing an infusion device in which the flow restrictor is in the form of a tortuous serpentine-formed channel established between two members. In the latter the flow resistance is selectable just as a bolus function is provided. Also U.S. Pat. No. 5,993,414 discloses an infusion device utilizing a tortuous path flow restrictor.

In a second variant an infusion device comprises a first cavity containing a drive fluid, a flow restrictor comprising a flow channel, a second cavity in fluid communication with the first cavity through the flow channel, and a drug reservoir containing the drug to be infused, where the second cavity and the drug reservoir is arranged such that the volume of the drug reservoir diminishes when the volume of the second cavity increases. Further, drive means for expelling the drive fluid from the first to the second cavity through the flow restrictor is provided, whereby drug is expelled from the drug reservoir. The force-transmitting interface between the second cavity and the drug reservoir could be described as a secondary pump actuated by the drive means. As appears, the drug flow rate will be determined by the pressure generated by the applied force, the flow resistance in the flow restrictor and the viscosity of the drive fluid. Advantages of the second variant are that the (delicate) drug does not have to be forced through the narrow flow restrictor and that a drive fluid having a high viscosity can be used thereby allowing a flow restrictor with a smaller flow resistance to be used which will normally be less expensive to manufacture. Examples of the second variant are disclosed in U.S. Pat. No. 2,605,765 and German published patent application 25 52 446.

In the above referred infusion devices using the bleeding hole principle, it has been an object to provide a constant infusion rate which has been achieved using force generating means providing a near-constant force, e.g. different forms of springs. However, it is also possible to use the bleeding hole principle in combination with flow rate controlling means as known from the infusion device described in EP 1 177 802, this infusion device comprising processor controlled valve means which opens and closes the drug flow generated using a bleeding hole pump.

An advantage of the bleeding hole principle is that it can be implemented in a relatively simple and thus inexpensive way, this lending itself to be utilized in devices in which cost is an important factor. An example of a type of device in which low manufacturing costs are of particular relevance would be a prefilled, disposable infusion device.

Consequently, when it is an object to provide an infusion device which can be manufactured cost-effectively, it would in many cases not be desirable to incorporate expensive components such as electronic control means which in combination with display means could be used to provide the user with information in respect of the infusion process, e.g. display means indicating that infusion is in progress or flow sensors providing information as to the amount of drug infused or left in the reservoir.

In this respect U.S. Pat. No. 2,605,765 discloses an infusion device comprising a transparent housing allowing the user to view the actual position of the piston expelling the drug. Although the housing is provided with a graduation it would be difficult for the user to identify that an infusion has just started, i.e. at a time when the piston has hardly moved. Correspondingly, it may be difficult at a quick glance to determine whether the piston is in its rearmost or foremost position and thereby is empty or full. The latter situation may be critical if a used, empty device is applied as thus the user would not receive the intended medication. Especially when used by older or otherwise disabled persons, this risk is considered not merely to be theoretical.

Thus, it is an object of the present invention to provide a delivery device with an improved user interface. It is further objects of the present invention to provide a delivery device with improved delivery functionality in respect of dosing accuracy, variability and reliability. Further objects and advantages of the present invention will be apparent from the below disclosure as well as from the description of exemplary embodiments.

### DISCLOSURE OF THE INVENTION

It is a first object of the present invention to provide a fluid delivery device of the type using a flow restrictor in combination with a drive fluid which provides safe and easy identification whether infusion has started, thereby providing improved safety, and which can be manufactured in a cost-effective manner.

More specifically, the present invention in accordance with the first object is based on the concept that the component or structure undergoing the greatest initial transformation or change when pump action is initiated would be the best candidates for detecting this invent.

Thus, in a first aspect the present invention provides a delivery device comprising a housing, a first cavity containing a drive fluid, a flow restrictor comprising a flow channel, a second cavity in fluid communication with the first cavity through the flow channel, a drug reservoir having in a situation of use an outlet means, where the second cavity and the drug reservoir is arranged such that the volume of the drug reservoir diminishes when the volume of the second cavity increases. The delivery device further comprises drive means for expelling the drive fluid from the first to the second cavity through the flow restrictor, whereby drug is expelled from the drug reservoir through the outlet. In accordance with the invention, the housing comprises a transparent portion allowing the content of the second variable volume cavity or the flow restrictor to be viewed from outside the delivery device, wherein the drive fluid is coloured (e.g. using a dye) for easy visual verification of its presence in the second variable volume cavity or the flow restrictor. As most liquid drugs are either transparent or milky (such as crystal-containing insulin) any “strong” colour such as red or blue may be used.

By this arrangement it is possible to identify the initial changes in the flow channel and/or the second cavity. For example, in a preferred embodiment the second cavity is substantially collapsed just as the flow channel preferably is substantially empty, this allowing even very small amounts of coloured drive fluid to be identified visually by the user. In case it is deemed necessary to provide the second cavity with an initial amount of a fluid (e.g. to fill any gaps which may otherwise exist between a flexible drug reservoir surrounded by the second cavity), this initial amount of fluid may be transparent. This would also apply in case the flow channel was pre-filled with a fluid, however, the volume of the flow channel will for most purposes be neglectable such that an initially air-filled flow channel will be acceptable. Indeed, a given infusion pump may also be designed to primarily indicate to the user that infusion has taken place and that the reservoir is empty.

The bleeding hole arrangement incorporating the indicating principle of the invention may be used in combination with different secondary pump arrangements.

In a preferred embodiment the delivery device comprises a reservoir cavity in which the drug reservoir is contained. The drug reservoir comprises a moveable portion, where the space external to the drug reservoir and between the moveable portion of the drug reservoir and the reservoir cavity defines the second cavity. A flexible membrane-member may be arranged within the reservoir cavity thereby dividing the reservoir cavity in the drug reservoir and the second cavity. In a further embodiment the reservoir cavity has a generally cylindrical form with a moveable piston being arranged within the reservoir cavity thereby dividing the reservoir cavity in the drug reservoir and the second cavity.

In a further preferred embodiment the infusion device comprises a drug reservoir defined within a generally flexible enclosure arranged within the reservoir cavity thereby dividing the reservoir cavity in the drug reservoir and the second variable volume cavity substantially surrounding the drug reservoir.

For the above secondary pump arrangements the drug reservoir in an initial state preferably takes up substantially the entire volume of the reservoir cavity, the second variable volume cavity in the initial state being substantially fully collapsed which would allow for easy detection of coloured drive entering the second cavity.

The outlet means may be adapted to be brought in fluid communication with external infusion means (e.g. a catheter tubing or transcutaneous access means such as an infusion needle, a flexible infusion cannula or a plurality of micro-penetrators) or may be supplied with these. In the latter case the fluid communication may be established just prior to use, before or after the drug delivery device has been arranged on the user.

An infusion (or delivery) device of the above type may also be manufactured or offered to the user as a system in which individual components are combined with each other to provide an aggregate device. For example, it may be desirable to offer a system comprising a disposable, pre-filled drug unit, a durable drive-force providing unit and a disposable unit comprising the drive fluid and the flow restrictor. For such a system different flow restrictors providing different infusion rates in combination with a given drive-force could be offered.

Correspondingly, in a second aspect the present invention provides a fluid transmitting device comprising a first variable volume cavity containing a drive fluid, a flow restrictor as discussed and described above, a second variable volume cavity in fluid communication with the first variable volume cavity through the flow channel.

Whereas it has been a first object of the present invention to provide a delivery device with an improved user interface, it is a further object of the present invention to provide a delivery device with improved delivery functionality in respect of dosing accuracy, variability and reliability.

As the drive fluid flow-rate in a bleeding hole pump depends on the pressure generated by the force acting on the drive fluid, it readily appears that for a constant flow of drug a constant force should be applied to the first cavity, however, some drive means does not provide such a constant force, this specifically applying to most spring arrangement, especially those utilizing a compressed helical coil as a drive means.

Thus, in a further aspect of the invention, a solution to this problem is provided by utilizing a piston which compensates for a non-constant force provided by the drive means by varying the effective area of a moveable piston acting on a fluid in an enclosure, thereby influencing the generated pressure. However, this arrangement may also be utilized to provide a desired non-constant drive fluid flow-rate on the basis on a constant or non-constant force acting upon such a piston.

More specifically, for general application a reservoir is provided, comprising a cavity member defining a cavity and having a first opening and a second opening, a piston being received in the first opening. The piston has a distal end facing towards the cavity, a proximal end facing away from the cavity, a longitudinal axis and a circumferential outer surface portion, the cross-sectional area of the piston varying along the longitudinal axis thereof, the piston being moveable along the longitudinal axis. A seal member is arranged between the cavity member and the piston, the seal member being adapted to seal the gap therebetween as the piston is moved along its longitudinal axis. By this arrangement an area surrounded by the seal member corresponding to a cross-sectional area of the piston defines an effective piston area for transmitting a pressure to a fluid contained within the cavity.

Depending on the type of seal member, the area on which an applied force will act may additionally be defined by a portion of the seal member. For example, in a first embodiment the seal member is in the form of a seal member arranged between the cavity member and the piston, the seal member (e.g. lip seal) comprising a flexible inner portion in sealing circumferential engagement with the outer surface portion of the piston, the inner (or edge) portion being adapted to sealingly engage the outer surface portion when the piston is moved along its longitudinal axis. By this arrangement the area surrounded by the inner portion of the seal and corresponding to a cross-sectional area of the piston defines an effective piston area for transmitting an applied pressure to a fluid contained within the cavity. Alternatively, the seal member may be of the rolling diaphragm type whereby the effective area will be determined by the rolling point of the convolution arranged in the gap between the cavity member and the piston. In addition to the effective area of the piston, the diaphragm will contribute to the area upon which an applied force will act.

The reservoir of the invention is intended for use in combination with a drive means providing a drive force acting upon and moving the piston towards the cavity, the piston thereby expelling a fluid contained within the cavity out through the second opening, wherein the drive means provides a given drive force as a function of the position of the piston along the longitudinal axis thereof, the combination of the drive force and the effective area providing a pressure within the cavity.

In case the drive means provides a constant drive force (e.g. utilizing a gas or gas-liquid-mixture as drive means), the cross-sectional area of the piston along its longitudinal axis can be configured to provide a given desired non-constant pressure in the cavity as a function of the position of the piston.

In case the drive means provides a drive force which varies as a function of the position of the piston, the cross-sectional area of the piston along its longitudinal axis can be configured to provide a constant pressure in the cavity as a function of the position of the piston, i.e. a constant function pressure. For such a varying drive force, the cross-sectional area of the piston along its longitudinal axis may be configured to provide a given desired non-constant pressure in the cavity as a function of the position of the piston.

In exemplary embodiments the drive means comprises a helical coil. In further exemplary embodiments the outer surface portion of the piston has a generally circular configuration along the longitudinal axis thereof, e.g. being fully or partially conical, however, in principle the piston may have any desired cross-function configuration as long as a proper sealing can be established. Also the cavity may have any desirable form, e.g. a cylindrical member which in combination with a generally circular piston may form a unit resembling a typical piston-cylinder arrangement. The seal member may be made from an elastomeric material. A lip seal may either be attached to or formed integrally with the cavity member, whereas a diaphragm may be formed as a separate member or integrally with the piston and/or the cavity member.

As the piston may be configured to provide a desired pressure as a function of the piston position, the piston may comprise one or more portions in which the cross-sectional area along the longitudinal axis in a direction away from the distal end either increases, decreases or is constant.

In an exemplary embodiment the reservoir is incorporated into a delivery device comprising a housing, a first variable volume cavity containing a drive fluid, a flow restrictor comprising a flow channel, a second variable volume cavity in fluid communication with the first variable volume cavity through the flow channel, and a variable volume drug reservoir having in a situation of use an outlet means. The second variable volume cavity and the variable volume drug reservoir are arranged such that the volume of the drug reservoir diminishes when the volume of the second cavity increases. The delivery device further comprises drive means for expelling the drive fluid from the first to the second cavity through the flow restrictor, whereby drug is expelled from the drug reservoir through the outlet, wherein the first variable volume cavity and the drive means are in the form of a reservoir and drive means as described above.

In another exemplary embodiment the reservoir is incorporated into a delivery device in which drug flow is controlled by directly expelling drug through a flow restrictor; the delivery device comprising a housing, a variable volume drug reservoir, a flow restrictor comprising a flow channel, and an outlet means in fluid communication with the first variable volume drug reservoir through the flow channel. A drive means is provided for expelling drug from the drug reservoir through the flow restrictor to the outlet means, wherein the variable volume drug reservoir cavity and the drive means are in the form of a reservoir and drive means as described above.

The outlet means may be adapted to be brought in fluid communication with infusion means (e.g. a catheter tubing or transcutaneous access means such as an infusion needle, a flexible infusion cannula or a plurality of micro-penetrators) or may comprise these. In the latter case the fluid communication between the drug reservoir and the outlet means may be established just prior to use, before or after the drug delivery device has been arranged on the user.

The above-described two specific applications are only exemplary as the principles of the compensating piston arrangement can be used in a varity of applications in which a driving force driving a piston has to be compensated or modified.

To further improve the reliability and dosing accuracy of a delivery device of the bleeding hole type using a drive fluid, the present invention also addresses the problems which may be associated with use of flow restrictors having very narrow flow channels which may be prone to obstruction in case the drive fluid is not entirely free from any impurities (e.g. particles or gas (air) bubbles) which may find their way to the flow restrictor and there result in full or partial obstruction.

Addressing this problem, the present invention provides a simple and cost-effective remedy to prevent or reduce the likelihood that impurities contained in the drive fluid will enter the flow restrictor.

Correspondingly, in a further aspect of the invention, a solution to this problem is provided by a delivery device comprising a housing, a first variable volume cavity containing a drive fluid, a flow restrictor comprising a flow channel, a second variable volume cavity in fluid communication with the first variable volume cavity through the flow channel, and a variable volume drug reservoir having in a situation of use an outlet. The second variable volume cavity and the variable volume drug reservoir are arranged such that the volume of the drug reservoir diminishes when the volume of the second cavity increases. The delivery device further comprises drive means for expelling the drive fluid from the first to the second cavity through the flow restrictor, whereby drug can be expelled from the drug reservoir through the outlet. To prevent undesired matter from entering the flow restrictor the first variable volume cavity comprises an occlusion restrictor in the form of an outlet portion with an outlet opening in fluid communication with the flow channel, the outlet portion protruding into the first variable volume cavity. In an exemplary embodiment the first variable volume cavity is defined by an interior wall surface, where the outlet portion comprises an outlet opening arranged at a distance from the interior wall surface surrounding the outlet portion.

The protruding outlet portion may have any desired configuration such as generally tubular with an outlet opening arranged at a distal end thereof. The outlet portion may also comprise a plurality of outlet openings, the most proximal thereof being arranged at a distance from the wall surface surrounding the outlet portion, however, in case some of the openings are very small, they may be arranged in the proximity of the wall surface thus serving as a filter element. Should such a filter opening occlude then drive fluid will be expelled through one or more of the openings arranged at a distance from the wall surface.

As a further means to reduce the likelihood that entrapped gas (air) bubbles will influence operation of the pump, the structure conducting fluid from the first variable volume cavity to the flow restrictor may be provided with venting means by which air bubbles passing by will be eliminated.

Thus, in a further aspect of the invention, a solution to this problem is provided by a delivery device comprising a housing, a first variable volume cavity containing a drive fluid, a flow restrictor comprising a flow channel, a second variable volume cavity in fluid communication with the first variable volume cavity through the flow channel, and a variable volume drug reservoir having in a situation of use an outlet. The second variable volume cavity and the variable volume drug reservoir are arranged such that the volume of the drug reservoir diminishes when the volume of the second cavity increases. The delivery device further comprises drive means for expelling the drive fluid from the first to the second cavity through the flow restrictor, whereby drug can be expelled from the drug reservoir through the outlet. To prevent from air bubbles from entering the flow restrictor venting means is arranged between the first variable volume cavity and the flow restrictor.

Advantageously the venting means comprises a membrane permeable to air but substantially impermeable to the fluid conducted through the structure, e.g. of Gore-Tex® type. The venting means may be used either alone or in combination with the protruding outlet portion.

If the fluid in the secondary cavity has a different thermal expansion than the reservoir itself, then temperature changes can cause the pump to deliver or suck drug. To partly or fully compensate for this effect, a compensation element made of one or more materials with a lower thermal expansion coefficient than the secondary reservoir (e.g. stainless steel or ceramic) may be comprised in the secondary reservoir.

Thus, in a yet further aspect of the invention, a solution to this problem is provided by a delivery device comprising a housing, a first variable volume cavity containing a drive fluid, a flow restrictor comprising a flow channel, a second variable volume cavity in fluid communication with the first variable volume cavity through the flow channel, and a variable volume drug reservoir having in a situation of use an outlet. The second variable volume cavity and the variable volume drug reservoir are arranged such that the volume of the drug reservoir diminishes when the volume of the second cavity increases. The delivery device further comprises drive means for expelling the drive fluid from the first to the second cavity through the flow restrictor, whereby drug can be expelled from the drug reservoir through the outlet. To compensate for temperature changes, the delivery device further comprises a fixed volume cavity formed from a first material, the variable volume drug reservoir being arranged there within and containing an initial amount of drug, the space between the drug reservoir and the fixed volume cavity forming the second variable volume cavity, the second variable volume cavity containing an initial amount of drive fluid, a compensation component made from a second material arranged within the second variable volume cavity, wherein the volume of the compensation component is selected such that the combined thermal volume variation of the compensation component, the drive fluid and the drug contained within the fixed volume cavity essentially matches the thermal volume variation of the fixed volume cavity itself.

The terms first and second materials as used herein also incorporate embodiments in which the cavity and/or compensation component are formed from more than one material, the cavity or compensation component thereby having aggregate thermal expansion characteristics.

Addressing the problem of providing a high degree of flow rate accuracy, a flow restrictor and methods of manufacturing thereof is provided which ensure a high degree of accuracy for the flow resistance.

More specifically, a flow restrictor is provided comprising a first member with a first surface portion and a second member with a second surface portion, an intermediate layer, having a thickness, arranged between the first and second members and comprising opposed first and second surfaces in engagement with the first respectively the second surface portions. A trace is formed in the intermediate layer through the thickness thereof, whereby a flow channel is formed by the intermediate layer and the first and second surface portions in combination. The flow restrictor further comprises inlet and outlet means in fluid communication with the flow channel thereby providing a fluid flow path therethrough. The inlet and outlet means may be provided as openings in the first and/or second members or simply by the flow channel opening to the surroundings.

By this sandwich arrangement a flow channel is provided having a well defined “height” determined by the thickness of the intermediate layer, whereby merely the width of the channel has to be controlled during forming of the trace in the intermediate layer.

Whereas the intermediate layer is relative thin, the “thickness” of the two surrounding members may vary in accordance with the actual configuration of the flow restrictor. For example, the flow restrictor may be manufactured as a flexible structure comprising three thin foil or membrane layers laminated together, e.g. in the form of an “endless” structure comprising a large number of individual flow restrictors adapted to be separated later in the manufacturing process, or the flow restrictor may be formed integrally with the item in combination with which it is to be used, e.g. housing member(s) may form one or both of the first and second members.

In accordance with exemplary methods of manufacturing the flow restrictor, at least one of the first and second surface portions may be formed from a different material than the intermediate layer, e.g. from a material having a higher melting point than the intermediate layer. In exemplary embodiments the portions of the first and second surface portions in engagement with the intermediate layer are generally planar, the intermediate layer having a uniform thickness.

The above-described three-layered flow restrictor may be manufactured using different methods.

For example, the intermediate layer may be bonded to the first member or may be supplied integrally formed on the first member (e.g. by extrusion or depositing techniques) after which a trace is formed in the intermediate layer through the thickness thereof without changing the configuration of the first surface portion. Thereafter the second member is bonded onto the intermediate layer to form a flow channel. To allow the trace to be formed solely in the intermediate layer, the first member (or the portion of the first member in contact with the intermediate layer) is formed from a different material than the intermediate layer. For example, the first member (or a portion thereof) may be formed from a material having a higher melting point than the intermediate layer, this allowing the trace to be formed by using trace-forming means selected from the group comprising: laser beam means, electron beam means or embossing with a heated mold. The term “different material” includes materials of the same type but which with altered properties, e.g. by surface treatment. An alternative means for forming a trace is by etching.

In an alternative manufacturing process the trace is formed in the intermediate layer before this is bonded to the first member, this allowing the trace to be formed independently of the material from which the latter is made, e.g. the same material may be used for both the intermediate layer and the first member.

In a further method of manufacturing a flow restrictor the trace is formed during a process in which a patterned intermediate layer is formed or deposited on the first member. The depositing procedure may be selected from the group comprising plating, printing and vapour depositing.

The different layers may be bonded to each other using any suitable technique such as adhesive bonding, melting or (ultrasonic) welding. Although not strictly a bonding technique, in the present context this term, also includes mechanical fastening or clamping. The materials for the individual layers/members (when not formed directly on the first member) may be selected from any suitable group of polymers or metal foils, however, especially for the first member, also glass or ceramic materials may be used.

The above-described flow restrictor may be used in combination with devices disclosed in this specification, or with any other device requiring a flow restrictor.

As used herein, the term “drug” or “medicament” is meant to encompass any drug-containing flowable medicament capable of being passed through a delivery means such as a hollow needle in a controlled manner, such as a liquid, solution, gel or fine suspension. There is essentially no limitation on the type of liquid drug which can be used with the invention other than to exclude those liquid drugs which would be inappropriate to deliver to the subject in an auto-mated fashion using the infusion device of the invention. Representative drugs include peptides, proteins, and hormones. In the description of the preferred embodiments reference will be made to the use of insulin. Correspondingly, the term “subcutaneous” infusion is meant to encompass any method of infusion into a subject.

### BRIEF DESCRIPTION OF THE DRAWINGS

In the following the invention will be further described with references to the drawings, wherein

FIG. 1 shows a perspective view of a first infusion device in an initial state,

FIG. 2 shows a perspective view of the infusion device of FIG. 1 in an actuated state,

FIG. 3 shows a “horizontal” cross-sectional view of the infusion device of FIG. 2,

FIG. 4 shows a first “vertical” cross-sectional view of the infusion device of FIG. 2,

FIG. 5 shows a second “vertical” cross-sectional view of the infusion device of FIG. 2,

FIG. 6A shows in detail a flow restrictor,

FIG. 6B is an exploded perspective view of a flow restrictor device,

FIG. 7 shows in detail an infusion needle,

FIG. 8 shows a “horizontal” cross-sectional view of an infusion device in an initial state,

FIG. 9 shows the infusion device of FIG. 8 in an actuated state,

FIG. 10 shows the infusion device of FIG. 8 in an empty state,

FIG. 11 shows a schematic representation of a second infusion device,

FIG. 12 shows a schematic representation of a further infusion device,

FIG. 13A shows a schematic representation of variable-diameter piston in combination with a first seal,

FIG. 13B shows a schematic representation of variable-diameter piston in combination with a second seal,

FIG. 14A shows a schematic representation of a cylinder and flow restrictor combination,

FIG. 14B shows the arrangement of FIG. 14A with the addition of an occlusion restrictor, and

FIG. 15 shows a schematic representation of a further infusion device.

### DESCRIPTION OF EXEMPLARY EMBODIMENTS

FIG. 1 shows a schematic representation of an embodiment of the invention. Correspondingly, the configuration of the different structures as well as there relative dimensions are intended to serve illustrative purposes only. This also applies to the other figures. When in the following terms as “upper”, “lower”, “right” and “left” or similar relative expressions are used, these only refer to the appended figures and not to an actual situation of use. In the same way the terms “horizontal” and “vertical” refer to planes parallel with respectively perpendicular to a lower surface of the device to be described. Further, like structures are indicated by like reference numerals.

More specifically, FIG. 1 shows an infusion device 1 comprising a housing 10 and there from protruding actuation button 20. The housing comprises an upper surface 2 and a lower surface 3 (not to be seen) adapted to be arranged against a skin surface of a user. The upper surface is provided with a transparent window 4 allowing the user to view a drug reservoir arranged within the housing. In FIG. 1 the infusion device has been arranged against the skin of a user and the actuation button has pressed into the housing by the user thereby actuating the infusion device as will be explained in detail below.

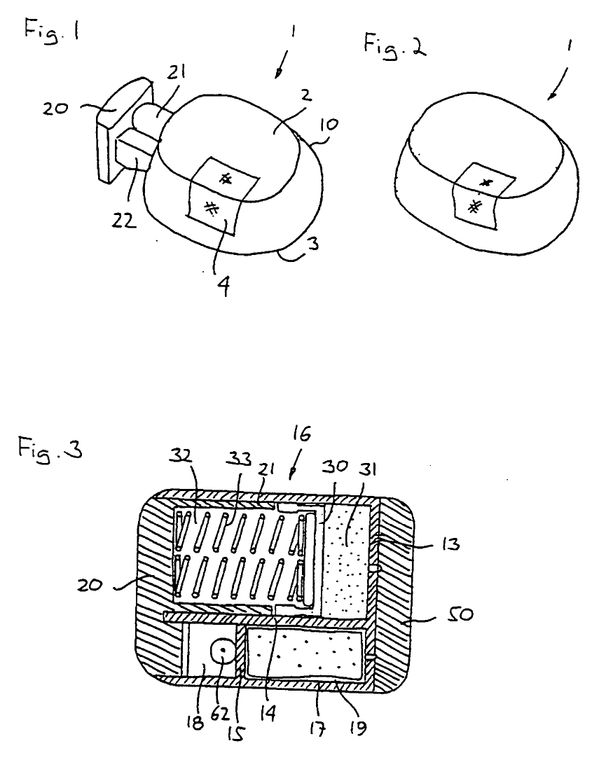

With reference to FIGS. 3-5 the general construction of the infusion device will be described. The housing comprises an upper wall 11, a lower planar base plate 12, side wall portions, an end wall 13 with an outer planar surface, and relative to the latter an opposed open end. Internally the housing comprises a first central wall 14 and a second oblique wall 15 in combination defining three compartments, a drive compartment 16, a reservoir compartment 17 and a needle compartment 18. The drive compartment forms a flat cylinder with an open proximal end and a substantially closed distal end. A piston 30 is slidingly arranged in the cylinder dividing the drive compartment in a distal fluid compartment 31 (corresponding to the above-described first cavity) filled with a coloured viscous drive fluid (e.g. silicon oil), and a proximal spring compartment 32. The actuation button 20 comprises a skirt portion 21 slidingly received in the cylinder thereby closing the spring compartment. In the spring compartment are arranged two helical compression springs 33 acting on the piston, however, any compressible material or member providing a spring action or any other means providing or generating a force (e.g. gas generating means or a liquid/gas mixture) acting on the piston may be utilized. The actuation button further comprises a wedge portion 22 to be received in the needle compartment.

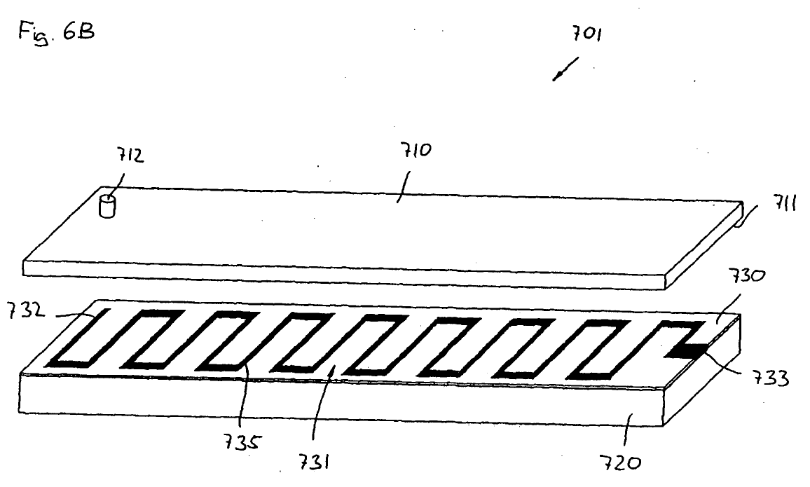

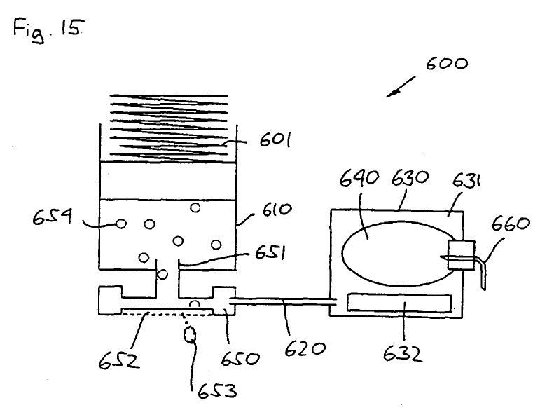

As best seen in FIG. 5 the reservoir compartment comprises a flexible drug reservoir 40 with an insulin-containing drug formulation. The reservoir is preferably manufactured from a transparent material allowing the user to view and control the drug through the window 4. In the initial state, i.e. before any drug has been expelled from the infusion device, the reservoir has a configuration substantially corresponding to the configuration of the reservoir compartment, thereby forming a neglectable cavity 19 (or dead-space) between the two components. In case an air filled dead space is not acceptable, the space may be filled with a fluid (for illustrative purposes is the gap between the reservoir and the reservoir compartment relatively large). As appears, the dead-space represents the above-described second cavity in a substantially fully collapsed state. Inside the drug reservoir is arranged a U-formed membrane element 41 formed from a self-sealing material and comprising upper and lower membrane portions 42, 43. In the end portion 13 is formed an outlet opening 34 from the fluid compartment and an inlet opening 44 to the reservoir compartment. The outlet opening may be provided with a portion protruding into the distal fluid compartment 31 (see the description of FIGS. 14B and 15 below).

The infusion device further comprises a flow restrictor member 50 (see FIG. 6A) comprising a planer surface 51 in which a serpentine trace 52 is formed between proximal and distal end portions 53, 54. The flow restrictor member 50 is bonded to the outer planar surface of the housing end portion with the proximal and distal end portions in register with the outlet 34 respectively the inlet openings 44. In this way a flow restrictor channel is formed between the two openings. Instead of the serpentine trace, any other structure providing the desired flow resistance may be used, e.g. a capillary tube or members with one or more small-diameter orifices formed therein. As appears, the resistance of the flow restrictor, the viscosity of the drive fluid and the force provided by the compressed springs will determine the rate at which the drive fluid will be forced through the flow restrictor to the reservoir compartment. When it is desirable to use the flow restrictor as an indicator means, the flow restrictor member or a portion thereof may be made form a transparent material allowing visual inspection of a coloured drive fluid located in the flow channel.

In the FIG. 6A embodiment a serpentine trace type flow restrictor was formed between two members, however, with reference to FIG. 6B an alternative configuration will be described.

More specifically, a flow restrictor device 701 comprises an upper member 710 with a generally planar lower surface 711 (cannot be seen in FIG. 6B), a lower member 720 with a generally planar upper surface (cannot be seen) and an intermediate layer 730 arranged on the upper surface of the lower member. The upper member comprises a through-going bore 712 serving as an inlet or outlet for the flow restrictor.

In the intermediate layer is formed a flow trace 731 having first and second end portions 732, 733 and a plurality of generally U-formed portions 735. The trace has a “height” (or “depth”) corresponding to the thickness of the intermediate layer and does thus not extend down into the lower member.

The intermediate layer may be provided as a foil or membrane member which is bonded to the upper surface of the lower member, and the trace may be formed either before or after the bonding; or the intermediate layer may be deposited onto the upper surface with the trace being formed either during or after the depositing procedure. Depending upon which of these manufacturing procedures are used, different materials and manufacturing techniques will be relevant, this as explained in detail in the above disclosure of the invention.

In an assembled state (not shown) the upper member 710 is attached (e.g. bonded) to the upper surface of the intermediate layer mating contact, whereby the flow trace will be “closed” to form a flow channel formed by the intermediate layer and the opposed surfaces of the upper and lower members in combination. The first end portion 732 of the flow channel is in communication with the opening whereas the second end portion opens directly to the surrounding space.

A first method for forming a flow restrictor as shown in FIG. 6B comprises the steps of (i) providing a first member comprising a first surface portion, a second member comprising a second surface portion and an intermediate layer having a thickness and comprising opposed first and second surfaces, the first surface portion being formed from a different material than the intermediate layer, (ii) bonding the first surface of the intermediate layer to the first surface portion, (iii) forming a trace in the intermediate layer through the thickness thereof without changing the configuration of the first surface portion, and (iv) bonding the second surface portion to the second surface of the intermediate layer, whereby a flow channel is formed by the intermediate layer and the first and second surface portions in combination.

A second method for forming a flow restrictor as shown in FIG. 6B comprises the steps of (i) providing a first member comprising a first surface portion and having an intermediate layer arranged thereon, the intermediate layer having a thickness and comprising a free surface, the first surface portion being formed from a different material than the intermediate layer, and a second member comprising a second surface portion, (ii) forming a trace in the intermediate layer through the thickness thereof without changing the configuration of the first surface portion, and (iii) bonding the second surface portion to the free surface of the intermediate layer, whereby a flow channel is formed by the intermediate layer and the first and second surface portions in combination.

A third method for forming a flow restrictor as shown in FIG. 6B comprises the steps of (i) providing a first member comprising a first surface portion, and a second member comprising a second surface portion, (ii) providing an intermediate layer having a thickness on the first surface portion, the intermediate layer having a free surface and a trace formed therein through the thickness thereof, without changing the configuration of the first surface portion, and (iii) bonding the second surface portion to the free surface of the intermediate layer, whereby a flow channel is formed by the intermediate layer and the first and second surface portions in combination.

The infusion device further comprises a hollow subcutaneous infusion needle 60 as shown in FIG. 7, comprising a distal pointed end 61 adapted to be introduced through a skin surface, a closed proximal end at which a needle wedge 62 is formed. In the body of the needle an opening 63 is formed in flow communication with interior of the needle. The proximal end of the needle is arranged in the needle compartment and with the needle body protruding through an opening 64 formed in the first wall 15 into the reservoir compartment and further into the reservoir. In the initial state (as supplied to the user and not shown in FIG. 5) the needle penetrates the upper membrane portion 42 with the distal end 61 arranged between the upper and lower membrane portions 42, 43 inside the reservoir.

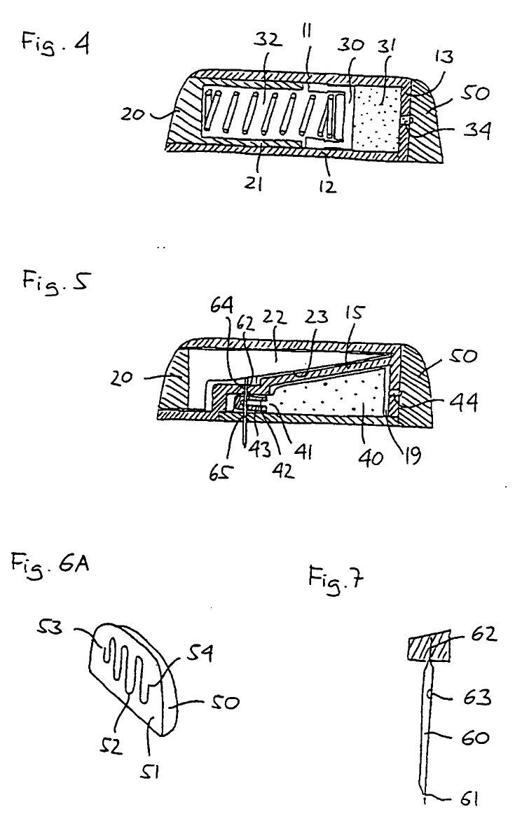

Next, with reference to FIGS. 4 and 5 actuation of the infusion device will be described. When the device has been positioned on a skin surface (preferably the lower surface comprises an adhesive coating) the user actuates the device by fully depressing the actuation button 20 until it locks in place in a recessed position (locking means arranged between the button and the housing is not shown in the figs.) whereby simultaneously the springs 33 are compressed and the wedge portion 22 is moved into the needle compartment. The wedge portion comprises a lower oblique surface 23 in sliding contact with the needle wedge 62 whereby the wedge portion forces the needle downwardly as it is pressed into housing. By this action the pointed distal needle end 61 penetrates the lower membrane portion 43 and is forced out through an opening 65 formed in the base portion. As the infusion device is attached to the skin surface of the user, the infusion needle is hereby introduced through the skin. When the needle is in its fully extended position, the needle opening 63 is positioned between the two membrane portions whereby a fluid communication is established from the drug reservoir to the user. At the same time the drive fluid starts to be expelled from the fluid compartment 31 and through the flow restrictor to the second cavity portion 19 of the reservoir compartment 17 where it gradually will compress the flexible reservoir and thereby force out the therein contained insulin-containing drug through the needle and into the user.

Initially air will be expelled from the needle just as air trapped in the flow restrictor and around the drug reservoir (if any) may result in an initial higher infusion rate, however, these effects will be neglectable.

In the shown embodiment the expelling means in form of springs 33 are “energized” during actuation of the device, however, to reduce the force needed to actuate the button 20 the spring means 33 may be pre-tensioned and the drive fluid 31 correspondingly pre-pressurized, whereby alone puncturing of the reservoir by the needle will actuate the expelling means and thereby start infusion.

Next, with reference to FIGS. 3 and 8-10 operation of the above-described infusion device will be described, the device comprising a coloured drive fluid which for illustrative purposes is “stronger” coloured than the drive fluid shown in FIGS. 1-7.

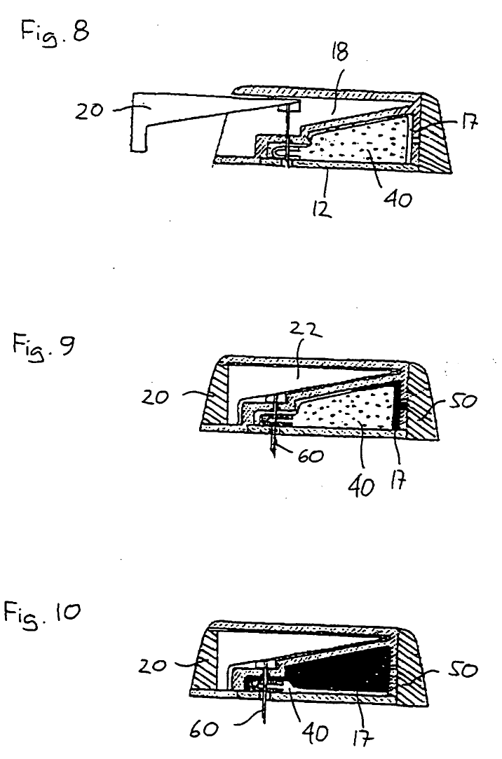

FIG. 8 shows an infusion device in an initial state corresponding to FIG. 1, i.e. the actuation button 20 has not yet been pressed into the needle compartment 18 and the reservoir 40 has an initial configuration substantially corresponding to the configuration of the reservoir compartment.

In FIG. 9 has the infusion device been attached to a skin surface of a user (not shown) and actuated by depressing the actuation button. As the needle is introduced subcutaneously by means of the wedge portion 22 a fluid communication is established between the user and the drug reservoir. At the same time the springs 33 is compressed whereby the drive fluid starts to be expelled from the fluid compartment 31 and through the flow restrictor to the second cavity portion of the reservoir compartment 17 where it gradually will compress the flexible reservoir and thereby force out the therein contained insulin-containing drug through the needle and into the user. By provision of the window 4 (see FIG. 1) the user will be able to detect the initial filling of the space between the reservoir and the walls of the surrounding compartment. In the shown embodiment is the lower surface of the reservoir attached to the base plate 12 which will cause the drive fluid to spread around the upper surface portions of the reservoir corresponding to the window 4. In this way a large area will become coloured early during filling of the second cavity. In addition, the flow restrictor member 50 may be manufactured from a transparent material allowing for even further detection of flow of liquid.

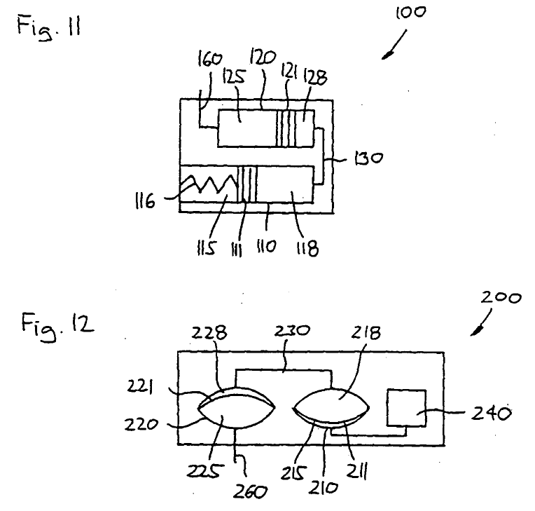

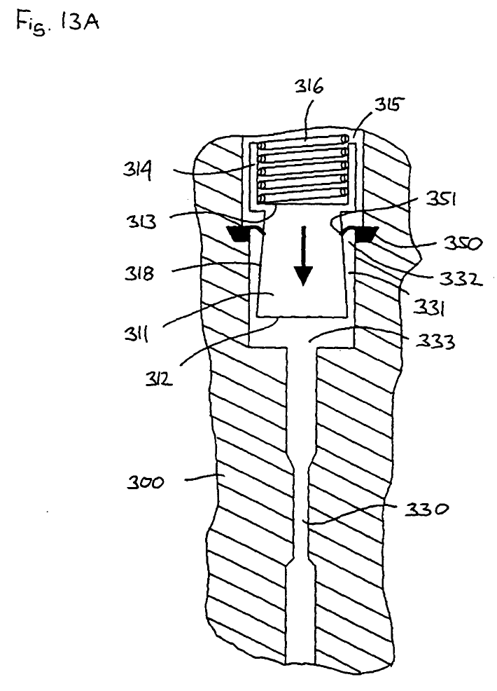

In FIG. 10 the reservoir compartment has been filled with drive fluid and the reservoir 40 has correspondingly been emptied, this resulting in an intensely coloured window 4 indicating to the user that the reservoir is empty or substantially empty. When the reservoir as shown is designed to be compressed from above towards the base plate, the “depth” of drive fluid as seen through the window will increase during infusion resulting in a more and more intense colour indicating to the user that infusion is in progress. However, this design does not provide a dosing read-out function and should correspondingly not be used as such.

Depending on the actual design of the infusion device and the arrangement of the flexible drug reservoir, it will be possible to utilize the colour indicating means in different ways. For example, when using a design as described above, the depressed actuation button will clearly indicate that the device has been actuated, however, the colour indicating means will provide the user with additional information as to the state of the infusion device, i.e. that infusion actually has started as indicated by the coloured drive fluid showing up in the flow channel and/or reservoir compartment. In case an actuation means is used which does not allow easy visual confirmation of the state, the colour indicating means may be used to simply indicate that an actual infusion device has been used and should be discarded.

The way the second cavity is filled with drug can be used in different ways depending on the actual design of the reservoir respectively the second cavity.

FIG. 11 shows a schematic representation of a second embodiment of an infusion device comprising first and second cylindrical compartments. The first cylindrical compartment 110 accommodates a first moveable piston 111 dividing the compartment in a drive means cavity 115 and a first cavity 118. The first cavity is filled with a viscous drive fluid and the drive means cavity comprises drive means in the form of a compressed spring 116 exerting a force on the first piston. The second cylindrical compartment 120 forms a reservoir cavity accommodating a second moveable piston 121 dividing the reservoir cavity in a drug reservoir 115 in flow communication with a subcutaneous needle 160 and a second cavity 128 in flow communication with the first cavity through a flow restrictor 130. As the drive fluid is transferred from the first to the second cavity through the flow restrictor, the second piston is forced towards the needle thereby expelling drug in a controlled fashion from the drug reservoir determined by the properties of the force applied on the first piston, the viscosity of the drive fluid and the resistance in the flow restrictor. When a transparent window is provided over the second reservoir cavity the coloured drive fluid makes it easy for the user to identify the position of the second piston and thereby to determine the amount of the drug infused or left.

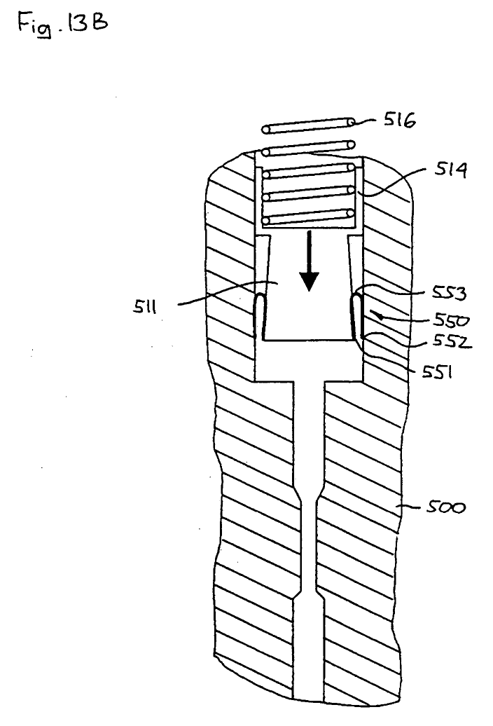

FIG. 12 shows a schematic representation of a further embodiment of an infusion device comprising first and second compartments. The first compartment 210 accommodates a first flexible membrane 211 dividing the compartment in a drive cavity 215 and a first cavity 218. The drive cavity is in flow communication with drive means 240 comprising a gas/fluid mixture which secures that the drive cavity is supplied with gas at a near-constant pressure thereby exerting a near-constant pressure on the first flexible membrane and thereby the first cavity filled with a viscous drive fluid. The second cylindrical compartment 220 forms a reservoir cavity accommodating a second flexible membrane 221 dividing the reservoir cavity in a drug reservoir 215 in flow communication with a subcutaneous needle 260 and a second cavity 228 in flow communication with the first cavity through a flow restrictor 230. As the drive fluid is transferred from the first to the second cavity through the flow restrictor, the second flexible membrane is forced towards the needle thereby expelling drug from the drug reservoir in a controlled fashion. When a transparent window is provided over the second cavity the coloured drive fluid makes it easy for the user to identify that infusion has started much in the same manner as was the case for the embodiment shown in FIGS. 8-10.

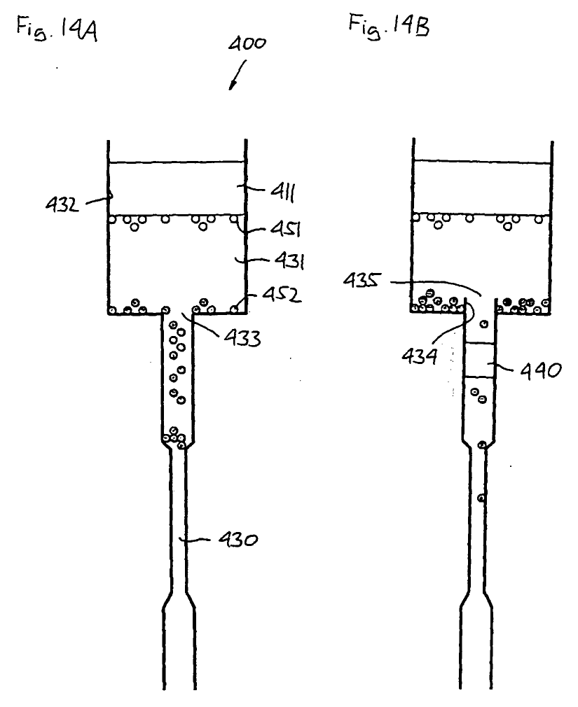

FIG. 13A shows a schematic representation of a portion of a delivery device of the above-described type (e.g. as shown in FIGS. 3 and 11), comprising a housing portion 300 forming a drive cavity 315 in which a helical spring 316 is arranged, a generally cylindrical drive fluid cavity 331 having a first proximal opening 332 in which a conical piston 311 is slidingly arranged (as indicated by the arrow), and a flow restrictor 330 in fluid communication with a second distal opening 333 of the fluid cavity. The piston comprises a distal end 312 facing towards the cavity, a proximal end 313 facing away from the cavity and comprising a skirt portion 314 for engaging the spring, a longitudinal axis and a circumferential outer surface portion 318. The piston has a generally conical shape with the cross-sectional area of the piston varying along the longitudinal axis thereof, i.e. diminishing from the distal end towards the proximal end. The piston is received in the first opening and can be moved along the longitudinal axis. A seal member 350 (here of the lip-type) is arranged between the housing portion and the piston corresponding to the first opening, the seal member comprising a flexible inner edge portion 351 in sealing engagement with the outer surface portion of the piston. The edge portion is adapted to sealingly engage the outer surface portion when the piston is moved along its longitudinal axis corresponding to its intended way of travel. By this arrangement the area surrounded by the edge portion and corresponding to a cross-sectional area of the piston defines an effective piston area for transmitting a pressure to a fluid contained within the cavity. In the shown embodiment the piston is guided linearly by the seal member and the spring, however, a portion of the piston (e.g. the skirt portion) may be adapted to slidingly engage the wall of the drive cavity.

When the force characteristics for the spring as well as the desired fluid pressure are known (both as a function of the piston position along its longitudinal axis) then the cross-sectional area of the piston along its length can be calculated. In the shown embodiment the spring delivers a constantly diminishing force as it expands, this being compensated by a correspondingly diminishing effective area of the piston corresponding to the portion surrounded by and in sealing engagement with the seal member, whereby a constant pressure in the fluid cavity is achieved. However, within certain limits (e.g. determined by the actual configuration of the seal and piston interface) a wide varity of desired pressure profiles can be provided for a given constant or non-constant spring characteristic.

FIG. 13B shows a schematic representation of an embodiment partially corresponding to the embodiment of FIG. 13A, however, instead of the lip-type seal member a convoluted rolling diaphragm 550 is used, the diaphragm comprising an inner circumferential portion 551 attached to the distal end 512 of the piston, an outer circumferential portion 552 attached to the drive fluid cavity, a circular convoluted “top” 553 being defined therebetween. Rolling diaphragms, either standard or custom designed, is offered by e.g. Marsh Bellofram Corporation, Newell, W. Va. Although the diaphragm will serve as a seal also in its “extended” states (e.g. non-convoluted), the shown embodiment is intended to be used with the diaphragm in its convoluted state. In this position, due to the pressure generated in the fluid, the diaphragm will be in close engagement with the opposed walls of the piston respectively the cavity over substantially the entire length in which position the effective pressure area of the system is defined by a diameter half-way between the hardware cylinder bore and piston diameters. Further, the skirt 514 is arranged in sliding engagement with the wall of the pressure cavity thereby controlling axial alignment of the piston during its travel.

FIG. 14A shows a schematic representation of a portion 400 of a delivery device of the above-described type (e.g. as shown in FIGS. 3 and 11), comprising a generally cylindrical drive fluid cavity 431 having a first proximal opening 432 in which a piston 411 is slidingly arranged, and a flow restrictor 430 in fluid communication with a second distal opening 433 of the drive fluid cavity. As appears, the second opening, and thereby the fluid entrance into the flow restrictor, is arranged flush with the interior wall surface of the cavity. The drive fluid cavity contains a viscous drive fluid (e.g. silicon oil) with a number of impurities in the form of air-bubbles 451 and particles 452. As shown, due to the flush arrangement of the second opening the particles may freely enter and eventually block the flow restrictor. In the shown situation the particles have collected at the “bottom” of the cavity and the air-bubbles have collected at the “top” of the cavity, however, during a normal situation of use the impurities may settle oppositely or may become more or less mixed in the highly viscous fluid.

FIG. 14B shows substantially the same arrangement as in FIG. 14B, however, in order to minimize the likelihood that impurities will enter the flow restrictor, the second opening has been provided with a tube-like extension 434 protruding into the drive fluid cavity, the actual second opening 435 being arranged at the distal free end portion of the extension. As the impurities during normal use conditions can be expected to collect primarily along the inner surfaces of the cavity, the arrangement of the second opening at a distance from the wall surface thus will minimize the likelihood that the impurities will enter the flow restrictor. In order to further reduce the risk of air bubbles entering the flow restrictor, the conduit portion between the fluid cavity and the flow restrictor is provided with venting means in the form of a tubular membrane portion 440, the membrane being permeable to air but substantially impermeable to the fluid conducted through the structure, e.g. of Gore™ type.

FIG. 15 shows a schematic representation of a delivery device embodying the same general drive principle as shown in the FIG. 3 embodiment. More specifically, the delivery 600 comprises a first variable volume cavity (or reservoir) 610 in the form a piston-cylinder arrangement (which may be of the type shown in FIGS. 13A and 13B) containing a drive (or primary) fluid with a number of air bubbles 654, a flow restrictor 620, a fixed volume cavity 630 in which a variable volume drug reservoir 640 in the form of a flexible pouch in fluid communication with an infusion needle 660 is arranged, the space between the pouch and the cavity forming a second variable volume cavity (or secondary reservoir) 641 in fluid communication with the first variable volume cavity through the flow restrictor. The drive means for expelling the drive fluid from the first to the second cavity through the flow restrictor is in the form of a helical spring 601. Between the first cavity and the flow restrictor a venting means 650 is arranged providing a fluid communication therebetween. The venting means is for illustrative purposes shown as a separate unit but it may advantageously be formed integrally with another structure. As also shown in FIG. 14B the venting means has an inlet portion 651 protruding into the first cavity as well as a Gore™ type membrane 652. By this arrangement the primary fluid is passed along the membrane to vent eventual air 653 out of the primary fluid. For maximum effect, the gap between the Gore™ membrane and the opposite surface should be so small that the air-bubbles actually get in contact with the membrane.

If the fluid in the secondary cavity has a different thermal expansion than the reservoir itself, then temperature changes can cause the pump to deliver or suck drug. To partly or fully compensate for this effect, a “compensation brick” 632 made of a material with a lower thermal expansion coefficient than the secondary reservoir (e.g. stainless steel or ceramic) is comprised in the secondary reservoir. If the volume of the brick is selected so the combined thermal expansion of the brick and the fluids in the secondary reservoir matches the thermal expansion of the reservoir itself, then the reservoir becomes temperature insensitive. For example, if the secondary reservoir is made of POM and contains 1 ml of silicone oil and 200 μl of insulin, then a stainless steel brick with a volume of 1.9 ml will be required to compensate for thermal expansion. Indeed, in case the thermal volume variation for the drive fluid and the drug differs greatly, the compensation will have to be determined as a compromise.

The venting means, the protruding inlet means and the compensation brick may be incorporated in a given device design independently of each other with corresponding effect.

In the above description of exemplary embodiments, the different structures providing the desired relations between the different components just as the means providing the described functionality for the different components (i.e. force generating means, flow restrictor, flexible reservoir etc.) have been described to a degree to which the concepts of the present invention will be apparent to the skilled reader. The detailed construction and specification for the different structures are considered the object of a normal design procedure performed by the skilled person along the lines set out in the present specification.

### DOCUMENTS CITED IN THE APPLICATION

U.S. Pat. No. 4,340,048
U.S. Pat. No. 4,552,561
U.S. Pat. No. 5,858,001
U.S. Pat. No. 6,280,148
U.S. Pat. No. 2,605,765
U.S. Pat. No. 5,957,895
U.S. Pat. No. 5,527,288
WO 02/15965
U.S. Pat. No. 5,993,414
U.S. Pat. No. 4,561,856
U.S. Pat. No. 4,744,786
U.S. Pat. No. 4,437,859
German published patent application 25 52 446
EP 1 177 802

1. A delivery device (1, 600) comprising:
a housing,
a first variable volume cavity (31, 610) containing a drive fluid,
a flow restrictor (50, 52, 430, 620) comprising a flow channel,
a second variable volume cavity (19, 631) in fluid communication with the first variable volume cavity through the flow channel,
a variable volume drug reservoir (40, 640) having in a situation of use an outlet means,
the second variable volume cavity and the variable volume drug reservoir being arranged such that the volume of the drug reservoir diminishes when the volume of the second cavity increases,
drive means (33, 601) for expelling the drive fluid from the first to the second cavity through the flow restrictor, whereby drug is expelled from the drug reservoir through the outlet means,
wherein the first variable volume cavity comprises an outlet portion (434, 651) with an outlet opening in fluid communication with the flow channel, the outlet portion protruding into the first variable volume cavity.

2. A delivery device as defined in claim 1, wherein the first variable volume cavity is defined by an interior wall surface, the outlet portion comprising an outlet opening arranged at a distance from the interior wall surface surrounding the outlet portion.

3. A delivery device as defined in claim 2, wherein the outlet portion (434, 651) has a generally tubular configuration with an outlet opening arranged at a distal end thereof.

4. A delivery device as defined in claim 2, wherein the outlet portion comprises a plurality of outlet openings, the most proximal thereof being arranged at a distance from the wall surface surrounding the outlet portion.

5. A delivery device as defined in claim 1, wherein venting means (440) is arranged between the first variable volume cavity and the flow restrictor.

6. A delivery device as defined in claim 5, wherein the venting means comprises means permeable to air but substantially impermeable to the drive fluid.

7. A delivery device (600) as defined in claim 1, further comprising
a fixed volume cavity (630) formed from a first material, the variable volume drug reservoir (640) being arranged there within and containing an initial amount of drug, the space between the drug reservoir and the fixed volume cavity forming the second variable volume cavity (631), the second variable volume cavity containing an initial amount of drive fluid,
a compensation component (632) made from a second material arranged within the second variable volume cavity, wherein
the volume of the compensation component is selected such that the combined thermal volume variation of the compensation component, the drive fluid and the drug contained within the fixed volume cavity essentially matches the thermal volume variation of the fixed volume cavity itself.

8. A delivery device (1, 500) as defined in claim 1, wherein the housing comprises a transparent portion allowing the content of the second variable volume cavity or the flow restrictor to be viewed from outside the device,
wherein the drive fluid is coloured for easy visual verification of its presence in the second variable volume cavity or the flow restrictor.

9. A delivery device as defined in claim 1, wherein the first variable volume cavity comprises:
a cavity member defining a cavity (331) and having a first opening (332) and a second opening (333),
a piston (311) having a distal end (312) facing towards the cavity, a proximal end (313) facing away from the cavity, a longitudinal axis and a circumferential outer surface portion (315), the cross-sectional area of the piston varying along the longitudinal axis thereof, the piston being received in the first opening and being moveable along the longitudinal axis,
a seal member (350, 550) arranged between the cavity member and the piston, the seal member being adapted to seal the gap therebetween when the piston is moved along its longitudinal axis,
whereby an area surrounded by the seal member corresponding to a cross-sectional area of the piston defines an effective piston area for transmitting a pressure to a fluid contained within the cavity.

10. A delivery device as defined in claim 1, wherein the flow restrictor (620, 701) comprises:
a first member (720) comprising a first surface portion and a second member (710) comprising a second surface portion,
an intermediate layer (730), having a thickness, arranged between the first and second members and comprising opposed first and second surfaces in engagement with the first respectively the second surface portions,
a trace (731) formed in the intermediate layer through the thickness thereof, whereby the flow channel is formed by the intermediate layer and the first and second surface portions in combination, and
inlet and outlet means in fluid communication with the flow channel thereby providing a fluid flow path therethrough.

11. A delivery device (600) comprising:
a housing,
a first variable volume cavity (610) containing a drive fluid,
a flow restrictor (620) comprising a flow channel,
a second variable volume cavity (631) in fluid communication with the first variable volume cavity through the flow channel,
a variable volume drug reservoir (640) having in a situation of use an outlet means,
the second variable volume cavity and the variable volume drug reservoir being arranged such that the volume of the drug reservoir diminishes when the volume of the second cavity increases,
drive means (601) for expelling the drive fluid from the first to the second cavity through the flow restrictor, whereby drug is expelled from the drug reservoir through the outlet means,
wherein venting means (652) is arranged between the first variable volume cavity and the flow restrictor.

12. A delivery device (600) comprising:
a housing,
a first variable volume cavity (610) containing a drive fluid,
a flow restrictor (620) comprising a flow channel,
a second variable volume cavity (631) in fluid communication with the first variable volume cavity through the flow channel,
a variable volume drug reservoir (640) having in a situation of use an outlet means,
the second variable volume cavity and the variable volume drug reservoir being arranged such that the volume of the drug reservoir diminishes when the volume of the second cavity increases,
drive means (601) for expelling the drive fluid from the first to the second cavity through the flow restrictor, whereby drug is expelled from the drug reservoir through the outlet means,
a fixed volume cavity (630) formed from a first material, the variable volume drug reservoir (640) being arranged there within and containing an initial amount of drug, the space between the drug reservoir and the fixed volume cavity forming the second variable volume cavity (631), the second variable volume cavity containing an initial amount of drive fluid,
a compensation component (632) made from a second material arranged within the second variable volume cavity, wherein
the volume of the compensation component is selected such that the combined thermal volume variation of the compensation component, the drive fluid and the drug contained within the fixed volume cavity essentially matches the thermal volume variation of the fixed volume cavity itself.

13. A delivery device (1, 600) comprising:
a housing,
a first variable volume cavity (31, 610) containing a drive fluid,
a flow restrictor (50, 52, 430, 620) comprising a flow channel,
a second variable volume cavity (19, 631) in fluid communication with the first variable volume cavity through the flow channel,
a variable volume drug reservoir (40, 640) having in a situation of use an outlet means,
the second variable volume cavity and the variable volume drug reservoir being arranged such that the volume of the drug reservoir diminishes when the volume of the second cavity increases,
drive means (33, 601) for expelling the drive fluid from the first to the second cavity through the flow restrictor, whereby drug is expelled from the drug reservoir through the outlet means,
wherein the housing comprises a transparent portion allowing the content of the second variable volume cavity or the flow restrictor to be viewed from outside the device, the drive fluid being coloured for easy visual verification of its presence in the second variable volume cavity or the flow restrictor.

14. A reservoir comprising:
a cavity member defining a cavity (331) and having a first opening (332) and a second opening (333),
a piston (311) having a distal end (312) facing towards the cavity, a proximal end (313) facing away from the cavity, a longitudinal axis and a circumferential outer surface portion (315), the cross-sectional area of the piston varying along the longitudinal axis thereof, the piston being received in the first opening and being moveable along the longitudinal axis,
a seal member (350, 550) arranged between the cavity member and the piston, the seal member being adapted to seal the gap therebetween when the piston is moved along its longitudinal axis,
whereby an area surrounded by the seal member corresponding to a cross-sectional area of the piston defines an effective piston area for transmitting a pressure to a fluid contained within the cavity.
